(19) United States
(12) Patent Application Publication
(10) Pub. No.: US 2011/0171203 A1
(43) Pub. Date: Jul. 14, 2011

(54) METHODS FOR TREATING NEOPLASTIC DISEASE TARGETING O-LINKED N-ACETYLGLUCOSAMINE MODIFICATIONS OF CELLULAR PROTEIN

(75) Inventors: Mauricio J. Reginato, Merion Station, PA (US); Keith Vosseller, Philadelphia, PA (US)

(73) Assignee: Philadelphia Health & Education Corporation d/b/a Drexel University College of Medicine, Philadelphia, PA (US)

(21) Appl. No.: 12/679,562

(22) PCT Filed: Oct. 3, 2008
(86) PCT No.: PCT/US08/78677

§ 371 Date: Jun. 1, 2010

Related U.S. Application Data
(60) Provisional application No. 60/977,290, filed on Oct. 3, 2007.

(51) Int. Cl. A61K 39/395; A61P 35/00; A61K 31/7088; C12Q 1/02; C12Q 1/68; C12Q 1/48

(52) U.S. Cl. 424/130.1; 514/44 A; 435/29; 435/6.14; 435/15; 435/6.13

(57) ABSTRACT

Methods for treating a neoplastic disease in a mammalian subject are provided. The method provides administering an inhibitor of O-GlcNAc transferase in an amount effective to reduce or eliminate the neoplastic disease in the mammalian subject. Method for diagnosing a risk factor for a neoplastic disease in a mammalian subject and methods for identifying a test compound which inhibits a hexosamine signaling pathway in a cell or tissue are provided.

### CROSS REFERENCE TO RELATED APPLICATIONS

This application claims the benefit of U.S. Application No. 60/977,290 filed Oct. 3, 2007, the entire disclosure of which is incorporated herein by reference.

### STATEMENT OF GOVERNMENT SUPPORT

This invention was made by government support by Grant No. BC062762 from the Department of Defense. The United States Government has certain rights in this invention.

### FIELD

The invention generally relates to methods for treating a neoplastic disease in a mammalian subject. The method provides administering an inhibitor of O-GlcNAc transferase in an amount effective to reduce or eliminate the neoplastic disease in the mammalian subject. The invention further relates to methods for assessing one or more a risk factor for a neoplastic disease in a mammalian subject and a method for identifying a test compound which inhibits a hexosamine signaling pathway in a cell or tissue.

### BACKGROUND

Breast cancer is the most common female cancer in the Western world, affecting 1 of 8 women over their lifetime. J. Harris et al., Diseases of the Breast, Lippincott Williams and Wilkins, Philadelphia, Pa. (1999). Understanding the mechanisms and signaling pathways that initiate and drive mammary oncogenesis is critical, as they may serve as critical markers for the early detection of breast cancer, and may be targeted in developing therapeutic interventions. The ERBB receptor tyrosine kinase is linked to development of many human cancers including breast cancer. Amplification of ERBB2 leading to overexpression of the receptor occurs in many epithelial-derived tumors including breast, ovarian, gastric and salivary cancers. T. Holbro et al., Exp Cell Res 284: 99-110 (2003). In about 10 to 30% of breast cancer cases, ERBB2 is amplified and these tumors tend to be aggressive with poor clinical outcome. M. F. Press et al., Prog Clin Biol Res 354A: 209-21, (1990). ERBB receptors are current targets of a number of therapies now used in the clinic, including humanized antibodies such as Herceptin which target ErbB2 function. R. Nahta et al., Cancer Lett 232: 123-38 (2006). However, only about one third of patients with ERBB2-overexpressing metastatic breast cancer respond to this therapy and the majority of cancers that initially respond to Herceptin begin to progress again within one year, indicating resistance to these agents. C. L. Vogel et al., J Clin Oncol 20: 719-26 (2002). In addition, studies using combination therapies targeting ERBB2 along with traditional chemotherapies have shown increased response and survival rates. R. Nahta et al., Cancer Lett 232: 123-38 (2006). Thus, identifying novel ways to target aggressive metastatic tumors is critical in developing future single and combination therapies that will increase both magnitude and duration of therapeutic benefit.

Ligand binding to ERBB receptors induces formation of receptor homo- and heterodimers and activation of intrinsic kinase domain, leading to phosphorylation of specific tyrosine residues which serve as docking sites to recruit multiple proteins leading to activation of intracellular signaling pathways that regulate many biological responses including cell growth, survival, migration, and changes in gene expression. M. A. Olayioye et al., Embo J 19: 3159-67 (2000). Two of the main pathways activated by ERBB receptors are the MEK-MAP kinase and the PI3K-AKT kinase pathway. In addition, ERBB2 activates the NF-κB pathway and specific downstream gene expression. M. Karin et al., Nat Rev Cancer 2: 301-10 (2002).

It has been established that ERBB2-mediated phosphorylation based signaling cascades contribute to inappropriate proliferative, survival, and metastatic signals related to cancer. B. P. Zhou et al., Semin Oncol 30: 38-48 (2003). Although relatively unknown, it is now clear that the cytosolic and nuclear serine/threonine post-translational carbohydrate modification O-linked N-acetylglucosamine (O-GlcNAc) acts as a regulatory switch mechanism in signaling. L. Wells et al., Science 291: 2376-8 (2001); G. W. Hart, Annu Rev Biochem 66: 315-35 (1997). Cytosolic and nuclear enzymes dynamically catalyze addition and removal of O-GlcNAc in response to various cell stimuli. S. P. Iyer et al., J Biol Chem 278: 24608-16 (2003). O-GlcNAc is found in all cell types examined on diverse functional classes of proteins and appears to be as widespread as phosphorylation. While site-specific regulatory roles for O-GlcNAc are only now being elucidated, it is clear that O-GlcNAc plays important roles in a variety of signaling processes, including heterotrimeric G protein receptor and tyrosine kinase receptor signaling. J. E. Kudlow, J Cell Biochem 98: 1062-75 (2006); R. Shafi et al., Proc Natl Acad Sci USA 97: 5735-9 (2000); Z. T. Kneass et al., J Biol Chem 280: 14579-85 (2005); K. Vosseller et al., Proc Natl Acad Sci USA 99: 5313-8 (2002). O-GlcNAc and O-phosphorylation sometimes compete for modification of specific serine/threonine residues, as in the case of c-myc and the estrogen receptor. K. Kamemura et al., Prog Nucleic Acid Res Mol Biol 73: 107-36 (2003); X. Cheng et al., Biochemistry 39: 11609-20 (2000). Such reciprocity would alter phosphorylation based signaling. Additionally, O-GlcNAc has been shown to effect proximal phosphorylation events. F. I. Comer et al., Biochemistry 40: 7845-52 (2001). Global reciprocity between O-GlcNAc and phosphorylation has been reported in cells. T. Lefebvre, Biochim Biophys Acta 1472: 71-81 (1999). Alternatively, O-GlcNAc may facilitate phosphorylation through mechanisms such as recruitment of kinases to specific complexes. Several signaling molecules in pathways implicated in oncogenic phenotypes have been shown to be modified by O-GlcNAc, including the p85 subunit of PI 3-kinase in endothelial cells and Akt in adipocytes. M. Federici et al., Circulation 106: 466-72 (2002); S. Y. Park et al., Exp Mol Med 37: 220-9. 2005. O-GlcNAc likely influences protein-protein interactions, and thus formation of specific protein complexes involved with ERBB2 signaling may be altered by modulation of O-GlcNAc, ultimately effecting transformed phenotypes dependent on ERBB2. Although it is now clear that O-GlcNAc contributes to regulation of signaling and there is a growing list of proteins modified by O-GlcNAc, there are no studies testing whether altering O-GlcNAc has negative effects on cancer phenotypes and whether it can alter ERBB2-mediated signaling in cancer models. M. D. Roos et al., Mol Cell Biol 17: 6472-80 (1997).

Pharmacologic inhibition of the transferase catalyzing addition of O-GlcNAc (OGT) or the enzyme which removes O-GlcNAc (O-GlcNAcase) are routes to modulate O-GlcNAc. B. J. Gross et al., J Am Chem Soc 127: 14588-9 (2005). Modulation of O-GlcNAc levels pharmacologically or genetically have been shown to alter growth/survival phenotypes linked to changes in cell cycle progression and alterations in phosphorylation patterns. N. O'Donnell et al., Mol Cell Biol 24: 1680-90 (2004); C. Slawson et al., J Biol Chem 280: 32944-56 (2005); G. Boehmelt et al., Embo J 19: 5092-104 (2000); W. Zhu et al., Embo J 20: 5999-6007 (2001). Thus, a proper balance of O-GlcNAc and phosphorylation appears to be required during proliferation/cell cycle progression, and O-GlcNAc may regulate important signals which are often dysregulated in cancer cells. PCT International Application WO 2006/092049; Hart et al., Nature 446: 1027-1032, 2007; Chou and Hart, Adv. Exp. Med. Biol., 491: 413-418, 2001; G. Boehmelt et al., Embo J 19: 5092-104 (2000); A. Brunet et al., Oncogene 9: 3379-87 (1994); F. I. Comer et al., Anal Biochem 293: 169-77 (2001); G. L. Corthals et al., Methods Enzymol 405: 66-81 (2005); J. Debnath et al., Cell 111: 29 (2002); B. J. Gross et al., J Am Chem Soc 127: 14588-9 (2005); C. Guerrero et al., Mol Cell Proteomics 5: 366-78 (2006); N. Lamarre-Vincent, J Am Chem Soc 125: 6612-3 (2003); M. S. Macauley et al., J Biol Chem 280: 25313-22 (2005); N. O'Donnell et al., Mol Cell Biol 24: 1680-90 (2004); K. B. Reddy et al., Int J Oncol 30: 971-5 (2007); M. J. Reginato et al., Mol Cell Biol 25: 4591-601 (2005); M. J. Reginato et al., Nat Cell Biol 5: 733-40 (2003); P. L. Ross et al., Mol Cell Proteomics 3: 1154-69 (2004); H. J. Schaeffer et al., Science 281: 1668-71 (1998); S. E. Seton-Rogers et al., Proc Natl Acad Sci USA 101: 1257-62 (2004); P. Sharma et al., J Biol Chem 277: 528-34 (2002); C. Slawson et al., J Biol Chem 280: 32944-56 (2005); C. Tagwerker et al., Mol Cell Proteomics 5: 737-48 (2006); M. A. Trakselis et al., Bioconjug Chem 16:741-50 (2005); K. Vosseller et al., Proteomics 5: 388-98 (2005); K. Vosseller et al., Mol Cell Proteomics (2006); K. Vosseller et al., Biochimie 83: 575-81; L. Wells et al., Mol Cell Proteomics 1: 791-804 (2002); B. Xu et al., J Biol Chem 274: 34029-35 (1999); W. Zhu et al., Embo J 20: 5999-6007 (2001).

A need exists in the art to identify therapies that target aggressive metastatic tumors. Therapeutic modalities which give rise to single and combination therapies that will increase both magnitude and duration of therapeutic benefit are greatly desired.

### SUMMARY

The present invention provides methods for treating neoplastic disease in a mammalian subject. The methods comprise administering to the subject an inhibitor of O-GlcNAc transferase (OGT) in an amount effective to reduce or eliminate neoplastic disease in the mammalian subject. The methods provided herein are based in part on the finding that neoplastic cells have elevated hexosamine signaling pathway which results in an increase in proteins modified by O-GlcNAc. The experimental results presented herein show that the methods for treating neoplastic disease comprise administering compositions that inhibit OGT and thus target and reduce O-GlcNAc modification of proteins in cancer cells and reduce anchorage independent growth of cells. Targeting O-GlcNAc modification of proteins by inhibiting OGT in cancer cells also reduces cell growth and cell invasion. Targeting O-GlcNAc modification of proteins has no effect on growth or differentiation of normal mammary epithelial cells.

A method for treating neoplastic disease in a mammalian subject is provided which comprises administering to the subject an inhibitor of O-GlcNAc transferase in an amount effective to reduce or eliminate the neoplastic disease in the mammalian subject. The neoplastic disease can be an epithelial-derived cancer, including but not limited to, breast cancer, ovarian cancer, gastric cancer, lung cancer, liver cancer, prostate cancer, skin cancer or salivary cancer. The inhibitor can include, but is not limited to, a small chemical molecule, an antibody, an antisense nucleic acid, short hairpin RNA, or short interfering RNA.

A method for diagnosing a risk factor for a neoplastic disease in a mammalian subject is provided which comprises contacting cells or tissue from the subject with an assay system for detecting a cellular component upregulated in a hexosamine signaling pathway in the cell or tissue, and comparing the upregulation of the hexosamine signaling pathway in the cell or tissue to normal function of the hexosamine signaling pathway in the cell or tissue. In one aspect, the upregulated hexosamine signaling pathway is measured as elevated UDP-GlcNAc levels in the cell or tissue, increased O-GlcNAc modification of proteins, or increased activity of O-GlcNAc transferase enzyme, or elevated levels of O-GlcNAc transferase gene expression or O-GlcNAc transferase protein in the cell or tissue. The assay system can include assay components including, but not limited to, an antibody or a nucleic acid. The neoplastic disease can be an epithelial-derived cancer, including but not limited to, breast cancer, ovarian cancer, gastric cancer, lung cancer, liver cancer, prostate cancer, skin cancer or salivary cancer.

A method for identifying a test compound which inhibits a hexosamine signaling pathway in a cell or tissue is provided which comprises contacting the test compound with a cell-based assay system comprising a cell expressing O-GlcNAc transferase, and capable of catalyzing N-acetylglucosamine attachment to a serine or threonine residue of a protein, and detecting an effect of the test compound on inhibition of O-GlcNAc transferase activity or O-GlcNAc transferase gene expression, effectiveness of the test compound in the assay being indicative of inhibition of the hexosamine signaling pathway activity in the cell or tissue. The cell can be a neoplastic cell. In a further aspect, the cell can include but is not limited to, a mammary epithelial cell expressing ErbB2, BT20 tumor cell, MDA-MB-231 tumor cell, MDA-MB-453 tumor cell, MDA-MB-468 tumor cell or SKBR3 tumor cell. In one aspect, inhibition of hexosamine signaling is measured by inhibition of a tumor cell phenotype. In a further aspect, the tumor cell phenotype is cell growth, colony formation in soft agar, or cell invasion. The test compound is assessed for inhibition of cell growth, colony formation, or cell invasion growth of the neoplastic cell, and the test compound does not inhibit cell growth or differentiation of a non-neoplastic cell. The neoplastic cell can be an epithelial tumor cell and the non-neoplastic cell can be an epithelial cell. The test compound includes, but is not limited to, a small chemical molecule, an antisense nucleic acid, short hairpin RNA, or short interfering RNA. The test compound effect on the neoplastic cell decreases cell proliferation markers PCNA or Ki-67, or increases CDK inhibitor p27, or a combination thereof.

### BRIEF DESCRIPTION OF THE DRAWINGS

FIG. 1 shows a schematic of tumor cells which upregulate glycolysis, resulting in increased glucose consumption.

FIG. 2 outlines that inhibiting O-GlcNAc modifications as a potential mechanism to target tumor cells.

FIG. 3 shows that breast cancer cells upregulate hexosamine signaling pathway.

FIG. 4 shows that breast cancer cells upregulate hexosamine signaling pathway.

FIG. 5 shows breast cancer cells upregulate hexosamine signaling pathway.

FIG. 6 shows targeting OGT inhibits anchorage-independent growth of cancer cells.

FIG. 7 shows targeting OGT inhibits MCF-10-NeuT-mediated invasion and proliferation.

FIG. 8 shows targeting OGT inhibits MCF-10-NeuT-mediated invasion and proliferation.

FIG. 9 shows OGT knockdown-mediated inhibition of proliferation is associated with induction of CDK inhibitor p27.MCF-10A-NeuT cells were transfected with control or OGT siRNA.

FIG. 10 shows OGT inhibitor (ST060266) blocks growth and invasion on MCF-10A-NeuT cells.

FIG. 11 shows OGT inhibitor (ST060266) blocks growth and invasion on MCF-10A-NeuT cells.

FIG. 12 shows targeting O-GlcNAc modifications has no effect on growth or differentiation of normal mammary epithelial cells.

FIG. 13 shows that targeting O-GlcNAc modifications has no effect on growth or differentiation of normal mammary epithelial cells.

### DETAILED DESCRIPTION

The present invention provides methods for treating neoplastic disease in a mammalian subject which comprises administering an inhibitor of O-GlcNAc transferase (OGT) in an amount effective to reduce or eliminate cancer in the mammalian subject. The methods provided herein are based on the finding that neoplastic cells have elevated a hexosamine signaling pathway which results in an increase in protein modification by O-GlcNAc. The results presented herein show that targeting O-GlcNAc modification of proteins by inhibiting OGT in cancer cells reduces anchorage independent growth. Targeting O-GlcNAc modification of proteins by inhibiting OGT in cancer cells also reduces cell growth and cell invasion. Targeting O-GlcNAc modification of proteins has no effect on growth or differentiation of normal mammary epithelial cells.

The present invention is based on the findings that modulation of O-GlcNAc levels is a valuable strategy to normalize inappropriate signaling in cancer transformation. Pharmacologic inhibition of the transferase catalyzing addition of O-GlcNAc (OGT) or the enzyme which removes O-GlcNAc (O-GlcNAcase) are routes to modulate O-GlcNAc. (9). Modulation of O-GlcNAc levels pharmacologically or genetically have been shown to alter growth/survival phenotypes linked to changes in cell cycle progression and alterations in phosphorylation patterns (23, 36) (1) (48). A proper balance of O-GlcNAc and phosphorylation appears to be required during proliferation/cell cycle progression, and O-GlcNAc may regulate important signals which are often dysregulated in cancer cells. Thus, identifying novel ways to target aggressive metastatic tumors is critical in developing future single and combination therapies that will increase both magnitude and duration of therapeutic benefit.

It is to be understood that this invention is not limited to particular methods, reagents, compounds compositions or biological systems, which can, of course, vary. It is also to be understood that the terminology used herein is for the purpose of describing particular aspects only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Hexosamine signaling pathway” refers to a dynamic cycle of addition and removal of O-linked N-acetylglucosamine (O-GlcNAc) at serine and threonine residues as a key regulator of nuclear and cytoplasmic protein activity. Like phosphorylation, protein O-GlcNAcylation dramatically alters the posttranslational fate and function of target proteins. Indeed, O-GlcNAcylation may compete with phosphorylation for certain Ser/Thr target sites. Like kinases and phosphatases, the enzymes of O-GlcNAc metabolism are highly compartmentalized and regulated. Yet, O-GlcNAc addition is subject to an additional and unique level of metabolic control. O-GlcNAc transfer is the terminal step in a “hexosamine signaling pathway” (HSP). In the HSP, levels of uridine 5′-diphosphate (UDP)-GlcNAc respond to nutrient excess to activate O-GlcNAcylation. Removal of O-GlcNAc may also be under similar metabolic regulation. Differentially targeted isoforms of the enzymes of O-GlcNAc metabolism allow the participation of O-GlcNAc in diverse intracellular functions. O-GlcNAc addition and removal are key to histone remodeling, transcription, proliferation, apoptosis, and proteasomal degradation. This nutrient-responsive signaling pathway also modulates important cellular pathways, including the insulin signaling cascade in animals and the gibberellin signaling pathway in plants. Alterations in O-GlcNAc metabolism are associated with various human diseases including diabetes mellitus and neurodegeneration. Love, D. C. and Hanover, J. A., Science STKE, 2005 (312): re13, 29 November 2005.

O-linked-N-acetylglucosamine (O-GlcNAc) is a regulatory post-translational modification of nuclear and cytosolic proteins. O-GlcNAc transferase (OGT) catalyzes the addition of O-linked N-acetylglucosamine (O-GlcNAc) at serine and threonine residues. O-GlcNAcase catalyzes the removal of O-GlcNAc at serine and threonine residues. These enzymes act as key regulators of nuclear and cytoplasmic protein activity. While only about 80 mammalian proteins have been identified to date that carry this modification, it is clear that this represents just a small percentage of the modified proteins. O-GlcNAc has all the properties of a regulatory modification including being dynamic and inducible. The modification appears to modulate transcriptional and signal transduction events. A working model is emerging that O-GlcNAc serves as a metabolic sensor that attenuates a cell's response to extracellular stimuli based on the energy state of the cell. Wells, et al., Biochem Biophys Res Commun. 302: 435-441, 2003.

“Patient”, “subject”, “vertebrate” or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

“Treating” or “treatment” includes the administration of the compositions, compounds or agents of aspects of the present invention to prevent or delay the onset of the symptoms, complications, or biochemical indicia of a disease, alleviating or ameliorating the symptoms or arresting or inhibiting further development of the disease, condition, or disorder (neoplastic disease, e.g., epithelial-derived cancer or breast cancer). “Treating” further refers to any indicia of success in the treatment or amelioration or prevention of the disease, condition, or disorder (neoplastic disease, e.g., epithelial-derived cancer or breast cancer), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of aspects of the present invention to prevent or delay, to alleviate, or to arrest or inhibit development of the symptoms or conditions associated with neoplastic disease The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of degenerative disease, e.g., neoplastic disease, e.g., epithelial-derived cancer or breast cancer, but does not yet experience or exhibit symptoms, inhibiting the symptoms of the neoplastic disease (slowing or arresting its development), providing relief from the symptoms or side-effects of neoplastic disease (including palliative treatment), and relieving the symptoms of neoplastic disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

“Inhibitors,” “activators,” and “modulators” of O-GlcNAc transferase activity in cells are used to refer to inhibitory, activating, or modulating molecules, respectively, identified using in vitro and in vivo assays for O-GlcNAc transferase activity binding or signaling, e.g., ligands, agonists, antagonists, and their homologs and mimetics.

“Modulator” includes inhibitors and activators. Inhibitors are agents that, e.g., bind to, partially or totally block stimulation, decrease, prevent, delay activation, inactivate, desensitize, or down regulate the activity of O-GlcNAc transferase, e.g., antagonists. Activators are agents that, e.g., bind to, stimulate, increase, open, activate, facilitate, enhance activation, sensitize or up regulate the activity of O-GlcNAc transferase, e.g., agonists. Modulators include agents that, e.g., alter the interaction of O-GlcNAc transferase with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring O-GlcNAc transferase, e.g., with altered activity, as well as naturally occurring and synthetic O-GlcNAc transferase, antagonists, agonists, small chemical molecules and the like. “Cell-based assays” for inhibitors and activators include, e.g., applying putative modulator compounds to a cell expressing a O-GlcNAc transferase activity and then determining the functional effects on hexosamine signaling, as described herein. “Cell based assays” include, but are not limited to, in vivo tissue or cell samples from a mammalian subject or in vitro cell-based assays comprising O-GlcNAc transferase that are treated with a potential activator, inhibitor, or modulator are compared to control samples without the inhibitor, activator, or modulator to examine the extent of inhibition. Control samples (untreated with inhibitors) can be assigned a relative O-GlcNAc transferase activity value of 100%. Inhibition of O-GlcNAc transferase is achieved when the O-GlcNAc transferase activity value relative to the control is about 80%, optionally 50% or 25-0%. Activation of O-GlcNAc transferase is achieved when the O-GlcNAc transferase activity value relative to the control is 110%, optionally 150%, optionally 200-500%, or 1000-3000% higher.

The ability of a molecule to bind to O-GlcNAc transferase can be determined, for example, by the ability of the putative inhibitor to bind to O-GlcNAc transferase immunoadhesin coated on an assay plate. Specificity of binding can be determined by comparing binding to a molecule other than O-GlcNAc transferase.

“Test compound” refers to any compound tested as a modulator of O-GlcNAc transferase. The test compound can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, test compound can be modulators that are genetically altered versions of O-GlcNAc transferase protein. Typically, test compounds will be small organic molecules, peptides, lipids, or lipid analogs.

A “naturally-occurring” nucleic acid molecule refers to an RNA or DNA molecule having a nucleotide sequence that occurs in nature (e.g., encodes a natural protein).

A “naturally-occurring” polypeptide or protein refers to a polypeptide molecule having an amino acid sequence that occurs in nature (e.g., encodes a natural protein).

“Gene” and “recombinant gene” refer to nucleic acid molecules which include an open reading frame encoding a O-GlcNAc transferase polypeptide, or mimetic, analog or derivative thereof, preferably a vertebrate, mammalian, bovine, human, avian reptilian, amphibian, osteichthyes, or chondrichthyes peptide, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one aspect, the language “substantially free” means a preparation of a O-GlcNAc transferase polypeptide, or mimetic, analog or derivative thereof, having less than about 30%, 20%, 10% and more preferably 5% (by dry weight), of non-O-GlcNAc transferase protein (also referred to herein as a “contaminating protein”). When the O-GlcNAc transferase polypeptide, or mimetic, analog or derivative thereof, or biologically active portion thereof, is recombinantly produced, it is also preferably substantially free of culture medium, i.e., culture medium represents less than about 20%, more preferably less than about 10%, and most preferably less than about 5% of the volume of the protein preparation. Aspects of the invention include isolated or purified preparations of at least 0.01, 0.1, 1.0, and 10 milligrams in dry weight.

A “non-essential” amino acid residue is a residue that can be altered from the wild-type sequence of the O-GlcNAc transferase polypeptide, or mimetic, analog or derivative thereof, without abolishing or more preferably, without substantially altering a biological activity, whereas an “essential” amino acid residue results in such a change. For example, amino acid residues that are conserved among the O-GlcNAc transferase polypeptides, or mimetic, analog or derivative thereof, those present in the domain of O-GlcNAc transferase polypeptide necessary for anti-apoptotic activity, are predicted to be particularly not amenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a O-GlcNAc transferase polypeptide, or mimetic, analog or derivative thereof, is preferably replaced with another amino acid residue from the same side chain family. Alternatively, in another aspect, mutations can be introduced randomly along all or part of a O-GlcNAc transferase polypeptide coding sequence, such as by saturation mutagenesis, and the resultant mutants can be screened for O-GlcNAc transferase biological activity to identify mutants that retain activity. Following mutagenesis of a O-GlcNAc transferase polypeptide, or mimetic, analog or derivative thereof, the encoded polypeptide can be expressed recombinantly and the activity of the protein can be determined.

“Biologically active,” when used in conjunction with O-GlcNAc transferase refers to a O-GlcNAc transferase polypeptide that affects regulation of hexosamine signaling in a manner substantially similar to that of full length O-GlcNAc transferase, and/or that activates hexosamine signaling in a mammalian subject.

A biologically active portion of O-GlcNAc transferase polypeptide can be a polypeptide which is, for example, 10, 25, 50, 100, 200, or more, amino acids in length. Biologically active portions of a O-GlcNAc transferase polypeptide can be used as targets for developing agents which modulate a O-GlcNAc transferase activity as described herein.

Calculations of homology or sequence identity (the terms are used interchangeably herein) between sequences are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred aspect, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence (e.g., when aligning a second sequence to the O-GlcNAc transferase polypeptide amino acid sequence, or mimetic, analog or derivative thereof, at least 10, preferably at least 20, more preferably at least 50, even more preferably at least 100 amino acid residues of the two sequences are aligned. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred aspect, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred aspect, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of aspects of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM 120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules encoding a modified O-GlcNAc transferase polypeptide, or mimetic, analog or derivative thereof, of aspects of the invention. BLAST protein searches can be performed with the XBLAST program, score=50, wordlength=3 to obtain amino acid sequences homologous to O-GlcNAc transferase polypeptide of aspects of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

Particular O-GlcNAc transferase polypeptide, or mimetic, analog or derivative thereof, in aspects of the present invention have an amino acid sequence sufficiently identical or substantially identical to the amino acid sequence of the O-GlcNAc transferase polypeptide. “Sufficiently identical” or “substantially identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as sufficiently or substantially identical.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An intact “antibody” comprises at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term antibody includes antigen-binding portions of an intact antibody that retain capacity to bind O-GlcNAc transferase polypeptide. Examples of binding include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the V_{L} and V_{H} domains of a single arm of an antibody, (v) a dAb fragment (Ward et al., Nature 341: 544-546, 1989), which consists of a VH domain; and (vi) an isolated complementarity determining region (CDR).

An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

“Fab antibodies” or “Fab fragments” refers to antibody fragments lacking all or part of an immunoglobulin constant region, and containing the Fab regions of the antibodies. Fab antibodies are prepared as described herein.

“Single chain antibodies” or “single chain Fv (scFv)” refers to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., Science 242: 423-426, 1988; and Huston et al., Proc. Natl. Acad. Sci. USA, 85: 5879-5883, 1988). Such single chain antibodies are included by reference to the term “antibody” fragments can be prepared by recombinant techniques or enzymatic or chemical cleavage of intact antibodies.

“Human sequence antibody” includes antibodies having variable and constant regions (if present) derived from human germline immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). Such antibodies can be generated in non-human transgenic animals, e.g., as described in PCT Publication Nos. WO 01/14424 and WO 00/37504. However, the term “human sequence antibody”, as used herein, is not intended to include antibodies in which CDR sequences derived from the germline of another mammalian species, such as a mouse, have been grafted onto human framework sequences (e.g., humanized antibodies).

Also, recombinant immunoglobulins may be produced. See, Cabilly, U.S. Pat. No. 4,816,567, incorporated herein by reference in its entirety and for all purposes; and Queen et al., Proc. Nat'l Acad. Sci. USA 86: 10029-10033, 1989.

“Monoclonal antibody” refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

“Polyclonal antibody” refers to a preparation of more than 1 (two or more) different antibodies to a O-GlcNAc transferase. Such a preparation includes antibodies binding to a range of different epitopes. Antibodies to O-GlcNAc transferase can bind to an epitope on O-GlcNAc transferase polypeptide so as to activate or mimic O-GlcNAc transferase activation of hexosamine signaling activity. These and other antibodies suitable for use in the present invention can be prepared according to methods that are well known in the art and/or are described in the references cited here. In preferred embodiments, anti-O-GlcNAc transferase polypeptide antibodies used in the invention are “human antibodies”—e.g., antibodies isolated from a human—or they are “human sequence antibodies”.

### High Throughput Assays For Modulators Of O-GlcNAc Transferase Gene Product

The compounds tested as modulators of O-GlcNAc transferase activity can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, modulators can be genetically altered versions of O-GlcNAc transferase. Typically, test compounds will be small organic molecules, peptides, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37: 487-493, 1991 and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g. , U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al., J. Amer. Chem. Soc. 114: 6568, 1992), nonpeptidal peptidomimetics with glucose scaffolding (Hirschmann et al., J. Amer. Chem. Soc. 114: 9217-9218, 1992), analogous organic syntheses of small compound libraries (Chen et al., J. Amer. Chem. Soc. 116: 2661, 1994), oligocarbamates (Cho et al., Science 261: 1303, 1993), and/or peptidyl phosphonates (Campbell et al., J. Org. Chem. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14: 309-314, 1996 and PCT/US96/10287), carbohydrate libraries (see, e.g., Liang et al., Science 274: 1520-1522, 1996 and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. No. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Candidate compounds are useful as part of a strategy to identify drugs for treating disorders including, but not limited to, neoplastic disease, e.g., epithelial-derived cancer or breast cancer. A test compound that binds to O-GlcNAc transferase or affects O-GlcNAc transferase gene expression is considered a candidate compound.

Screening assays for identifying candidate or test compounds that bind to O-GlcNAc transferase, or modulate the activity of O-GlcNAc transferase proteins or polypeptides or biologically active portions thereof, are also included in the invention. The test compounds can be obtained using any of the numerous approaches in combinatorial library methods known in the art, including, but not limited to, biological libraries; spatially addressable parallel solid phase or solution phase libraries; synthetic library methods requiring deconvolution; the “one-bead one-compound” library method; and synthetic library methods using affinity chromatography selection. The biological library approach can be used for, e.g., peptide libraries, while the other four approaches are applicable to peptide, non-peptide oligomer or small chemical molecule libraries of compounds (Lam, Anticancer Drug Des. 12: 145, 1997). Examples of methods for the synthesis of molecular libraries can be found in the art, for example in: DeWitt et al., Proc. Natl. Acad. Sci. U.S.A. 90: 6909, 1993; Erb et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and Gallop et al., J. Med. Chem. 37: 1233, 1994. In some embodiments, the test compounds are activating variants of O-GlcNAc transferase.

Libraries of compounds can be presented in solution (e.g., Houghten, Bio/Techniques 13: 412-421, 1992), or on beads (Lam, Nature 354: 82-84, 1991), chips (Fodor, Nature 364: 555-556, 1993), bacteria (U.S. Pat. No. 5,223,409), spores (U.S. Pat. Nos. 5,571,698, 5,403,484, and 5,223,409), plasmids (Cull et al., Proc. Natl. Acad. Sci. USA 89: 1865-1869, 1992) or on phage (Scott et al., Science 249: 386-390, 1990; Devlin, Science 249: 404-406, 1990; Cwirla et al., Proc. Natl. Acad. Sci. USA 87: 6378-6382, 1990; and Felici, J. Mol. Biol. 222: 301-310, 1991).

The ability of a test compound to modulate the activity of O-GlcNAc transferase or a biologically active portion thereof can be determined, e.g., by monitoring the inhibition or activation of iron uptake into cells in the presence of the test compound. Modulating the activity of O-GlcNAc transferase or a biologically active portion thereof can be determined by measuring inhibition or activation of iron uptake into cells. The binding assays can be cell-based or cell-free.

The ability of a test compound to modulate the activity of O-GlcNAc transferase and inhibit hexosamine signaling in cells can be determined by one of the methods described herein or known in the art for determining direct binding. In one embodiment, the ability of the O-GlcNAc transferase to bind to or interact with hexosamine signaling can be determined by monitoring inhibition of neoplastic cell growth.

This invention further pertains to novel agents identified by the above-described screening assays and uses thereof for treatments as described herein.

In one embodiment the invention provides soluble assays using O-GlcNAc transferase, or a cell or tissue expressing O-GlcNAc transferase gene product, either naturally occurring or recombinant. In another embodiment, the invention provides solid phase based in vitro assays in a high throughput format, where O-GlcNAc transferase is attached to a solid phase substrate via covalent or non-covalent interactions. Any one of the assays described herein can be adapted for high throughput screening.

In the high throughput assays of the invention, either soluble or solid state, it is possible to screen up to several thousand different modulators or ligands in a single day. This methodology can be used for O-GlcNAc transferase in vitro, or for cell-based or membrane-based assays comprising O-GlcNAc transferase gene product or O-GlcNAc transferase protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as toll-like receptors, transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I, 1993. Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, polyethylene glycol linkers are available from Shearwater Polymers, Inc. Huntsville, Ala.. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85: 2149-2154, 1963 (describing solid phase synthesis of, e.g., peptides); Geysen et al., J. Immun. Meth. 102: 259-274, 1987 (describing synthesis of solid phase components on pins); Frank & Doring, Tetrahedron 44: 6031-6040, 1988 (describing synthesis of various peptide sequences on cellulose disks); Fodor et al., Science 251: 767-777, 1991; Sheldon et al., Clinical Chemistry 39: 718-719, 1993; and Kozal et al., Nature Medicine 2: 753-759, 1996 (all describing arrays of biopolymers fixed to solid substrates). Non-chemical approaches for fixing tag binders to substrates include other common methods, such as heat, cross-linking by UV radiation, and the like.

### Modulating Or Inhibiting Expression Of And Transcripts

The invention further provides for nucleic acids complementary to (e.g., antisense sequences to) the nucleic acid sequences of the invention. Antisense sequences are capable of modulating or inhibiting the transport, splicing or transcription of protein-encoding genes, e.g., O-GlcNAc transferase-encoding nucleic acids. The modulation or inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind gene or message, in either case preventing or inhibiting the production or function of the protein. The association can be through sequence specific hybridization. Another useful class of inhibitors includes oligonucleotides which cause inactivation or cleavage of protein message. The oligonucleotide can have enzyme activity which causes such cleavage, such as ribozymes. The oligonucleotide can be chemically modified or conjugated to an enzyme or composition capable of cleaving the complementary nucleic acid. One can screen a pool of many different such oligonucleotides for those with the desired activity.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript of a phosphatase polypeptide of the invention. The term “RNAi” stands for RNA interference. This term is understood in the art to encompass technology using RNA molecules that can silence genes. See, for example, McManus, et al. Nature Reviews Genetics 3: 737, 2002. In this application, the term “RNAi” encompasses molecules such as short interfering RNA (siRNA), microRNAs (mRNA), small temporal RNA (stRNA). Generally speaking, RNA interference results from the interaction of double-stranded RNA with genes.

A. Antisense Oligonucleotides

The invention provides antisense oligonucleotides capable of binding O-GlcNAc transferase messenger RNA which can inhibit polypeptide activity by targeting mRNA. Strategies for designing antisense oligonucleotides are well described in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho, Methods Enzymol. 314: 168-183, 2000, describing an RNA mapping assay, which is based on standard molecular techniques to provide an easy and reliable method for potent antisense sequence selection. See also Smith, Eur. J. Pharm. Sci. 11: 191-198, 2000.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata, Toxicol Appl Pharmacol. 144: 189-197, 1997; Antisense Therapeutics, ed. Agrawal, Humana Press, Totowa, N.J., 1996. Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold, J. of Biol. Chem. 270: 13581-13584, 1995).

B. siRNA

“Small interfering RNA” (siRNA) refers to double-stranded RNA molecules from about 10 to about 30 nucleotides long that are named for their ability to specifically interfere with protein expression through RNA interference (RNAi). Preferably, siRNA molecules are 12-28 nucleotides long, more preferably 15-25 nucleotides long, still more. Preferably 19-23 nucleotides long and most preferably 21-23 nucleotides long. Therefore, preferred siRNA molecules are 12, 13, 14, 15, 16, 17, 18, 19, 20, 21, 22, 23, 24, 25, 26, 27 28 or 29 nucleotides in length.

RNAi is a two-step mechanism. Elbashir et al., Genes Dev., 15: 188-200, 2001. First, long dsRNAs are cleaved by an enzyme known as Dicer in 21-23 ribonucleotide (nt) fragments, called small interfering RNAs (siRNAs). Then, siRNAs associate with a ribonuclease complex (termed RISC for RNA Induced Silencing Complex) which target this complex to complementary mRNAs. RISC then cleaves the targeted mRNAs opposite the complementary siRNA, which makes the mRNA susceptible to other RNA degradation pathways.

siRNAs of the present invention are designed to interact with a target ribonucleotide sequence, meaning they complement a target sequence sufficiently to bind to the target sequence. The present invention also includes siRNA molecules that have been chemically modified to confer increased stability against nuclease degradation, but retain the ability to bind to target nucleic acids that may be present.

C. Inhibitory Ribozymes

The invention provides ribozymes capable of binding message which can inhibit polypeptide activity by targeting mRNA, e g., inhibition of polypeptides with O-GlcNAc transferase activity. Strategies for designing ribozymes and selecting the protein-specific antisense sequence for targeting are well described in the scientific and patent literature, and the skilled artisan can design such ribozymes using the novel reagents of the invention.

Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but can also be formed in the motif of a hairpin, hepatitis delta virus, group I intron or RnaseP-like RNA (in association with an RNA guide sequence). Examples of such hammerhead motifs are described by Rossi, Aids Research and Human Retroviruses 8: 183, 1992; hairpin motifs by Hampel, Biochemistry 28: 4929, 1989, and Hampel, Nuc. Acids Res. 18: 299, 1990; the hepatitis delta virus motif by Perrotta, Biochemistry 31: 16, 1992; the RnaseP motif by Guerrier-Takada, Cell 35: 849, 1983; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

### Method Of Treatment

The compositions of the present invention, e.g., inhibitors of O-GlcNAc transferase, are useful in the treatment of cancers. Specific cancers to be treated with the compositions of the invention include neoplastic disease or epithelial derived neoplastic disease, for example, estrogen negative breast cancer, estrogen positive breast cancer, prostate cancers (including androgen-independent prostate cancer), ovarian cancer, bladder cancer, brain cancer, head and neck cancer, kidney cancer, lung cancers such as small cell lung cancer and non-small cell lung cancer, myeloma, neuroblastoma/glioblastoma, pancreatic cancer, skin cancers, liver cancers, melanoma, colon cancer, cervical carcinoma, and leukemia, retinoblastoma, pancreatic islet carcinoma, or other epithelial-derived cancers.

In one embodiment, a method of treating or preventing the development of cancer in a mammalian subject comprising treating cancer cells of said subject with an O-GlcNAc transferase inhibitor composition of the invention is contemplated, either in vivo or ex vivo. The pharmaceutical compositions of the present invention may be administered to a subject via one or more routes to contact the cancer cells, as desired. For example, the compositions may be administered via oral, topical, systemic, enteral, parenteral (e.g., formulated for injection via the intravenous, intramuscular, subcutaneous, intracutaneous, or even intraperitoneal routes (e.g. by drip infusion)), subcutaneous, intra-portal, intra-prostatic, intramuscular, intra-venous, intra-arterial, intra-dermal, intra-thecal, intra-lesional, intra-tumoral, intra-bladder, intra-vaginal, intra-ocular, intra-rectal, intra-pulmonary, intra-spinal, transdermal, and subdermal routes. Further, the compositions may be delivered via placement within cavities of the body, regional perfusion at the site of a tumor or other desired location, nasal inhalation, pulmonary inhalation, impression into skin and electrocorporation. The route(s) of administration will vary according to the cell(s), tissue(s), organ(s), or system(s) to be treated.

In a further embodiment, the therapeutic compositions are delivered transdermally or by sustained release through the use of a transdermal patch containing the composition and an optional carrier that is inert to the compound, is nontoxic to the skin, and allows for delivery of the compound for systemic absorption into the blood stream. Such a carrier can be a cream, ointment, paste, gel, or occlusive device. The creams and ointments can be viscous liquid or semisolid emulsions. Pastes include absorptive powders dispersed in petroleum or hydrophilic petroleum. Further, a variety of occlusive devices can be utilized to release the active reagents into the blood stream and include semi-permeable membranes covering a reservoir contain the active reagents, or a matrix containing the reactive reagents.

The use of sustained delivery devices can be desirable, in order to avoid the necessity for the patient to take medications on a daily basis. “Sustained delivery” refers to delaying the release of an active agent, i.e., a compound of the invention, until after placement in a delivery environment, followed by a sustained release of the agent at a later time. A number of sustained delivery devices are known in the art and include hydrogels (U.S. Pat. Nos. 5,266,325; 4,959,217; 5,292,515), osmotic pumps (U.S. Pat. Nos. 4,295,987 and 5,273,752 and European Patent No. 314,206, among others); hydrophobic membrane materials, such as ethylenemethacrylate (EMA) and ethylenevinylacetate (EVA); bioresorbable polymer systems (International Patent Publication No. WO 98/44964 and U.S. Pat. Nos. 5,756,127 and 5,854,388); and other bioresorbable implant devices composed of, for example, polyesters, polyanhydrides, or lactic acid/glycolic acid copolymers (U.S. Pat. No. 5,817,343). For use in such sustained delivery devices, the compounds of the invention can be formulated as described herein. See, U.S. Pat. Nos. 3,845,770; 3,916,899; 3,536,809; 3,598,123; and 4,008,719.

The methods of this invention involve administering an inhibitor of O-GlcNAc transferase to a mammalian subject in effective amounts to reduce or eliminate neoplastic disease in the mammalian subject, while minimizing adverse impacts on non-cancer cells of the patient. Dosages of the compounds and compositions of the present invention vary with the particular compositions employed, the route of administration, the severity of the symptoms presented, the particular subject being treated, and the subjects other medications and treatment, as well as the subject's medical history. Precise dosages for oral, parenteral, nasal, or intrabronchial administration can be determined by the administering physician based on experience with the individual subject treated. An effective therapeutic dosage will contain a dosage sufficient to induce apoptosis of cancer cells. In one embodiment, the dosage of composition of the present invention is such that administration will decrease heoxosamine signaling pathway in the cell or tissue.

The amount of the compound of the invention present in each effective dose is selected with regard to consideration to the half-life of the compound, the identity and/or stage of the cancer, the patient's age, weight, sex, general physical condition and the like. The amount of active component or compound required to induce an effective apoptotic effect on cancer cells without significant adverse side effects varies depending upon the pharmaceutical composition employed and the optional presence of other components. Suitable dosages of compositions used to treat cancers as described herein can range from 1.0 μg to 500 mg compound(s)/kg patient body weight. In one embodiment, the dosage is at least 10 μg/kg. In another embodiment, the dosage is at least 100 μg/kg. In another embodiment, the dosage is at least 500 μg/kg. In another embodiment, the dosage is at least 1 mg/kg. In another embodiment, the dosage is at least 10 mg/kg. In another embodiment, the dosage is at least 50 mg/kg. In another embodiment, the dosage is at least 100 mg/kg. In another embodiment, the dosage is at least 250 mg/kg. In another embodiment, the dosage is at least 400 mg/kg. In another embodiment, the dosage is at least 500 mg/kg. In another embodiment, each dose will contain between about 5 pg peptide/kg patient body weight to about 10 mg/kg. Generally, a useful therapeutic dosage is between 1 to 5 mg peptide/kg body weight. Another embodiment of a useful dosage may be about 500 μg/kg of peptide. Other dosage ranges may also be contemplated by one of skill in the art. For example, dosages of the peptides of this invention may be similar to the dosages discussed for other cancer therapeutics.

Initial doses of a composition of this invention may be optionally followed by repeated administration for a duration selected by the attending physician. Dosage frequency may also depend upon the factors identified above, and may range from 1 to 6 doses per day for a duration of about 3 days to a maximum of no more than about 1 week. The compositions of this invention may also be administered as a continuous infusion for about 3-5 days, the specific dosage of the infusion depending upon the half-life of the compound. The compounds of this invention may also be incorporated into chemotherapy protocols, involving repetitive cycles of dosing. Selection of the appropriate dosing method would be made by the attending physician.

The following examples of specific embodiments for carrying out the present invention are offered for illustrative purposes only, and are not intended to limit the scope of the present invention in any way.

### Exemplary Embodiments

The methods provided herein are based on the finding that neoplastic cells have elevated a hexosamine signaling pathway which results in an increase in protein modification by O-GlcNAc. The results presented herein show that targeting O-GlcNAc modification of proteins by inhibiting OGT in cancer cells reduces anchorage independent growth. Targeting O-GlcNAc modification of proteins by inhibiting OGT in cancer cells also reduces cell growth and cell invasion. Targeting O-GlcNAc modification of proteins has no effect on growth or differentiation of normal mammary epithelial cells. Thus, identifying novel ways to target aggressive metastatic tumors is an important step in developing future single and combination therapies that will increase both magnitude and duration of therapeutic benefit.

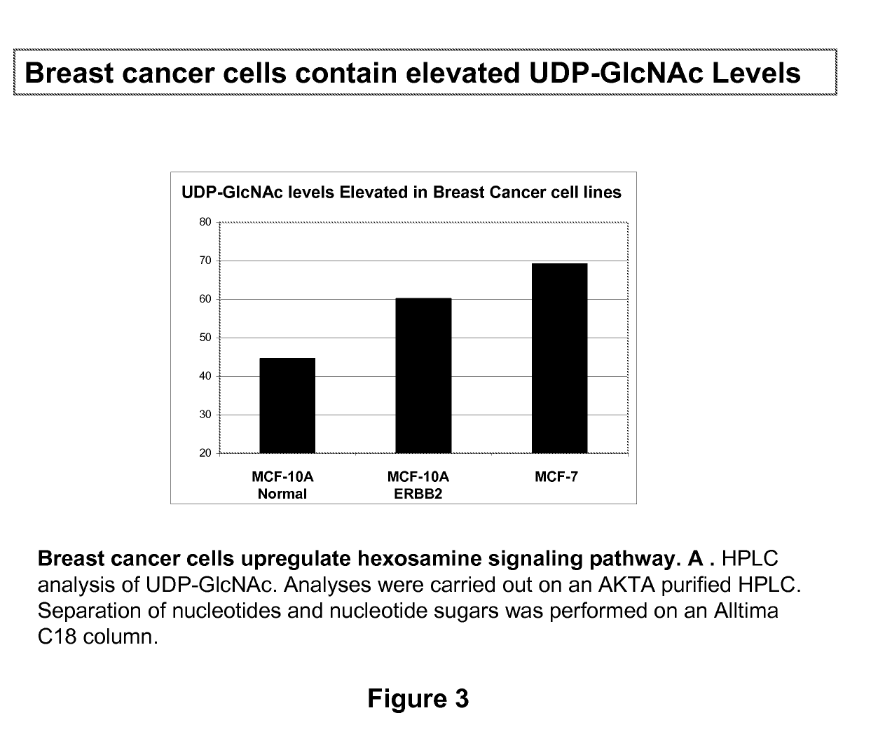

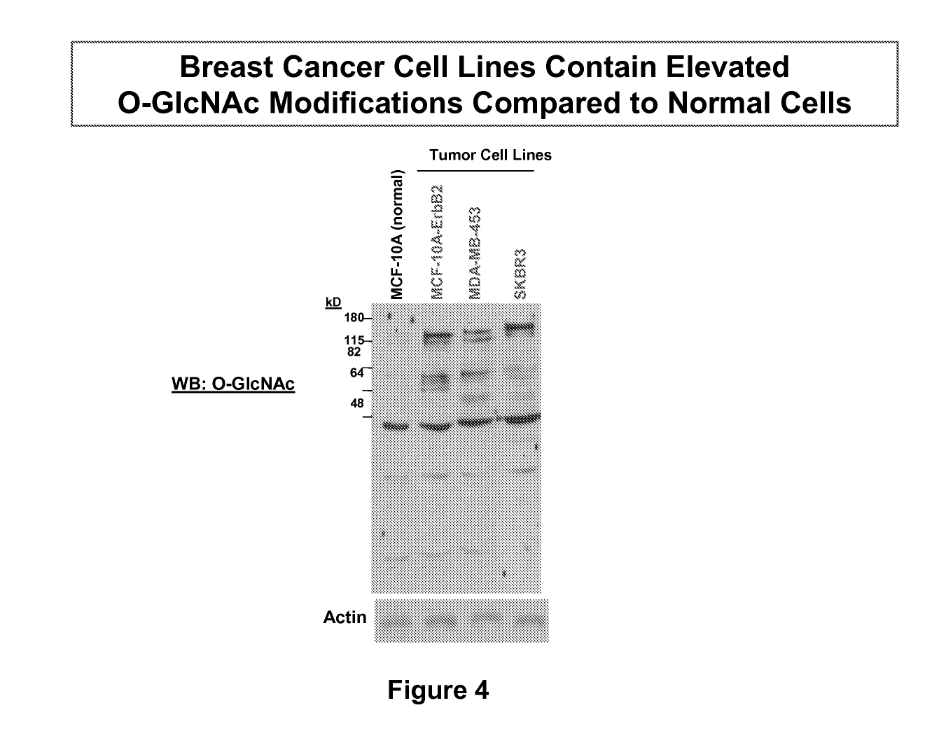

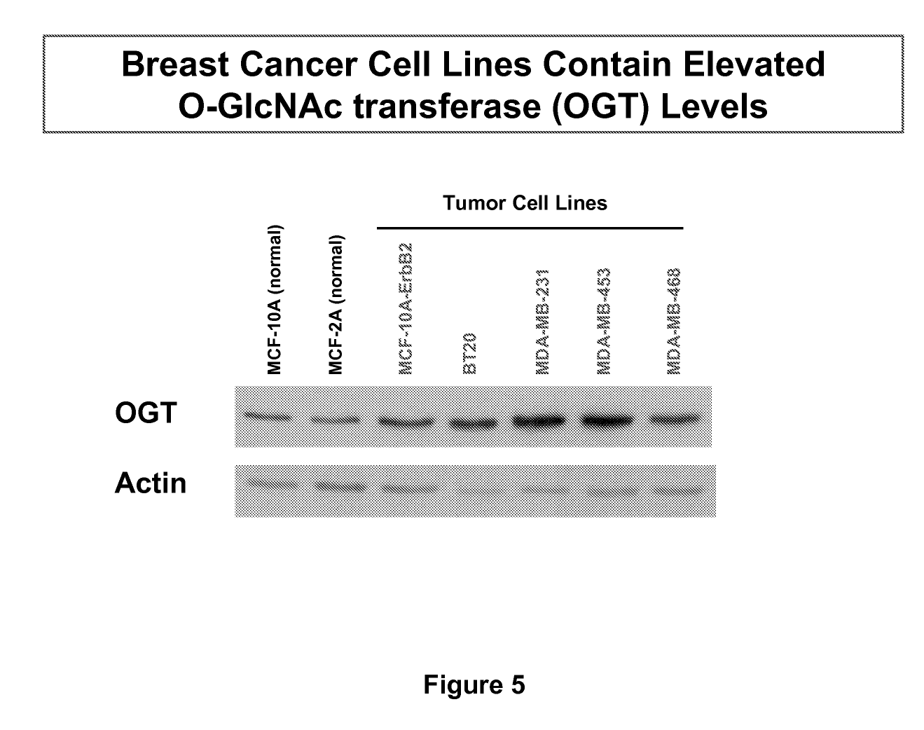

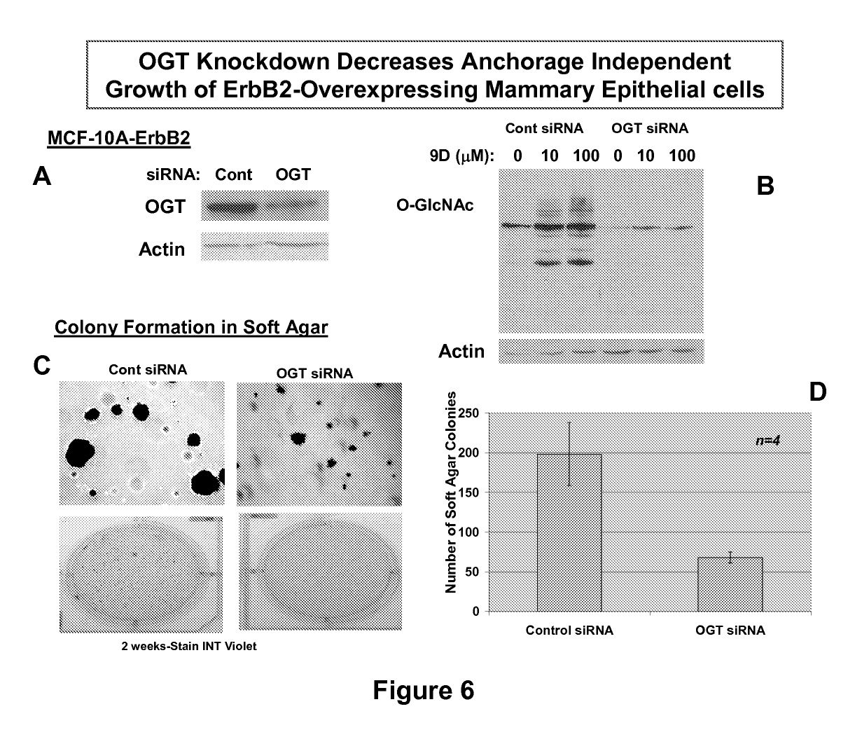

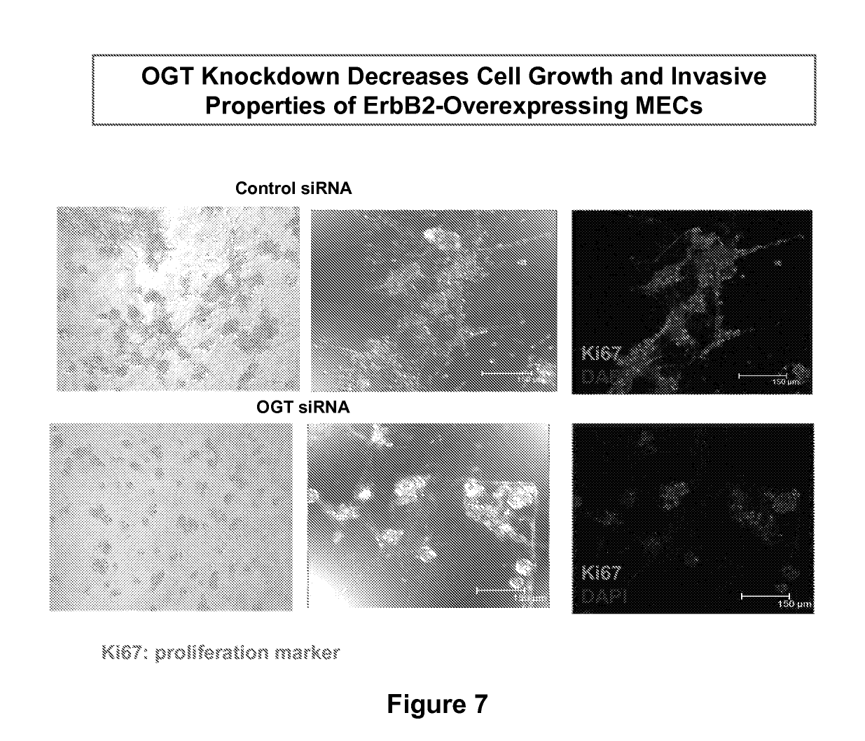

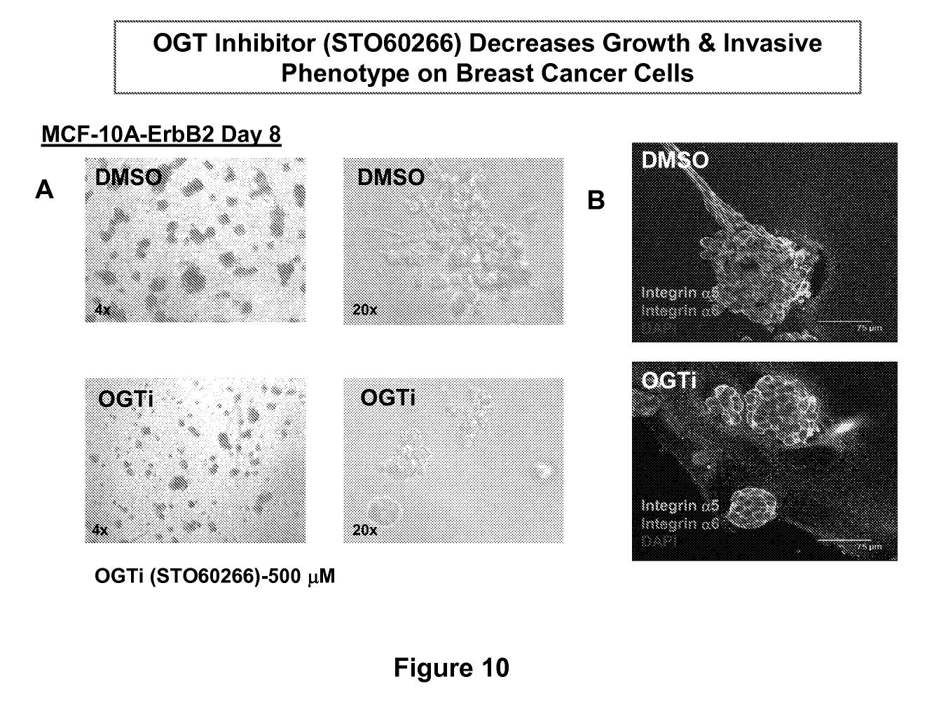

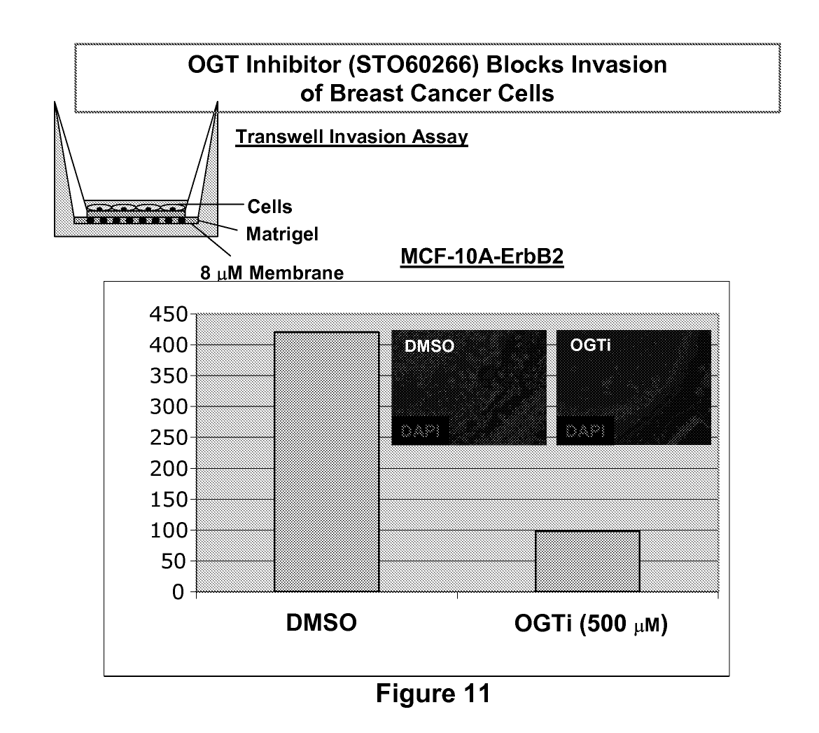

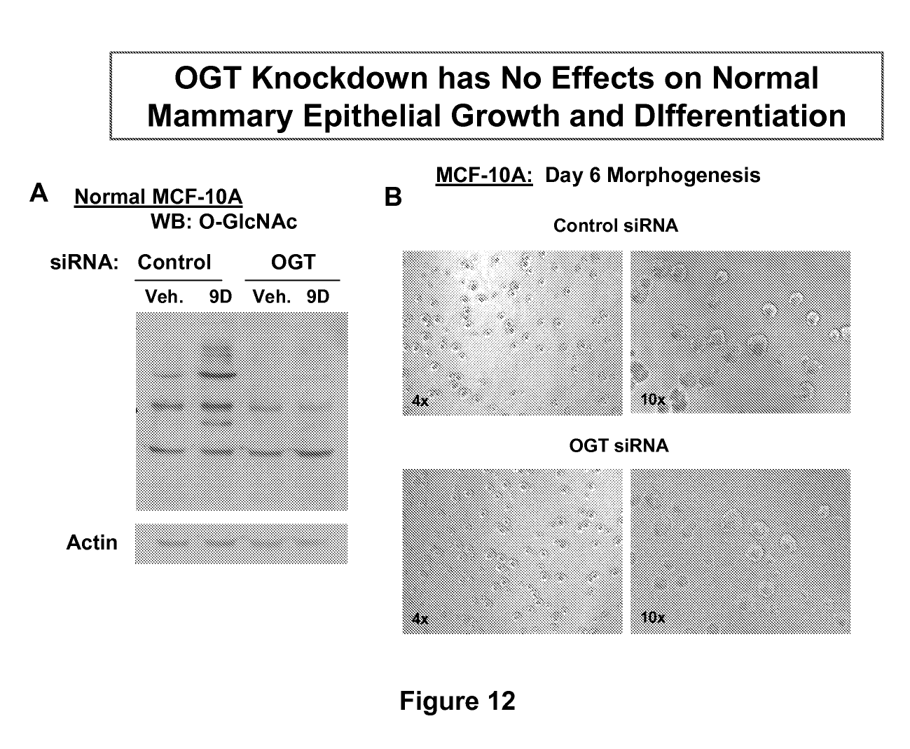

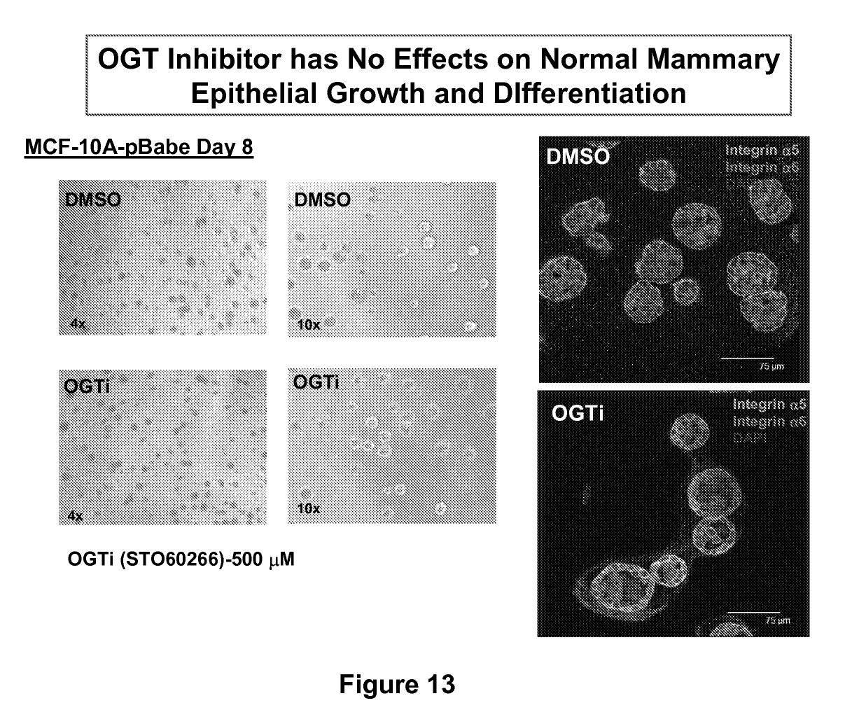

The present studies demonstrate that normal immortalized human mammary epithelial MCF-10A cells overexpressing oncogenic ERBB2, as well as breast cancer cells MCF-7, contain elevated levels of UDP-GlcNAc levels compared to normal mammary epithelial cells (MCF-10A) (FIG. 3). We also show that ERBB2 overexpressing cells and breast cancer cell lines that overexpress ERBB2 (SKBR3 and MDA-MB-453) have higher levels of O-GlcNAc modifications when compared to control MCF-10A cells (FIG. 4). Moreover, breast cancer cells compared to normal cells overexpress the O-GlcNAc transferase (OGT) (FIG. 5). In this application we show that inhibiting O-GlcNAc modifications, by two independent mechanisms, blocks breast cancer phenotypes in vitro. We targeted OGT via RNAi to specifically lower O-GlcNAc modifications in MCF-10A cells overexpressing ERBB2. We tested the effects of OGT RNAi by examining effects on OGT levels (FIG. 6A) and global O-GlcNAc levels in cells treated with or without the O-GlcNAcase inhibitor 9D (causes an elevation of O-GlcNAc). Control RNAi transfected cells (MCF-10A-ERBB2) displayed a significant increase in OGlcNAc modifications in the presence of inhibitor 9D (FIG. 6B) as expected. However, cells transfected with RNAi against OGT displayed significant decrease of basal O-GlcNAc modification and completely blocked 9D-mediated elevation of O-GlcNAc modifications (FIG. 6B) verifying the loss of OGT function. These cells were then placed in soft agar assays. Our data shows a four-fold decrease in colony formation in soft agar in OGT targeted cells (FIGS. 6C and 6D). We also examined effects of targeting OGT in cancer cells placed in 3 dimensional (3D) culture assays. Under 3D conditions, targeting OGT with RNAi caused dramatic inhibition of ERBB2-mediated oncogenic phenotypes including decreased cell growth and cell invasion compared to control RNAi cells (FIG. 6A). Knockdown of OGT in cancer cells lead to a two-fold inhibition of Ki67-positive staining (FIGS. 7, 8, and 9). Ki67 is a critical marker for proliferating cells. The inhibition of proliferation by targeting OGT led to induction of cell cycle inhibitor protein p27 (FIG. 7). The cancer inhibiting phenotype by targeting OGT was verified independently by targeting OGT using a specific OGT inhibitor (ST060266, TimTec LLC). Treating cancer cells with OGT inhibitor also blocked growth and cell invasion when cells were placed in 3D culture assay (FIG. 10). Using transwell invasion assay, we show a four-fold inhibition of invasion of cancer cells treated with OGT inhibitor (FIG. 11). Importantly, we show that targeting OGT in normal mammary epithelial cells (MCF-10A parental) is not toxic. We targeted OGT via RNAi in normal MCF-10A cells (FIG. 12) and treated them with OGT inhibitor (FIG. 13) and found no cytotoxic effect on cell growth and differentiation in 3D conditions. Thus the present studies show, by using two independent mechanisms, that lowering O-GlcNAc modifications in cancer cells significantly reduces oncogenic phenotypes in vitro.

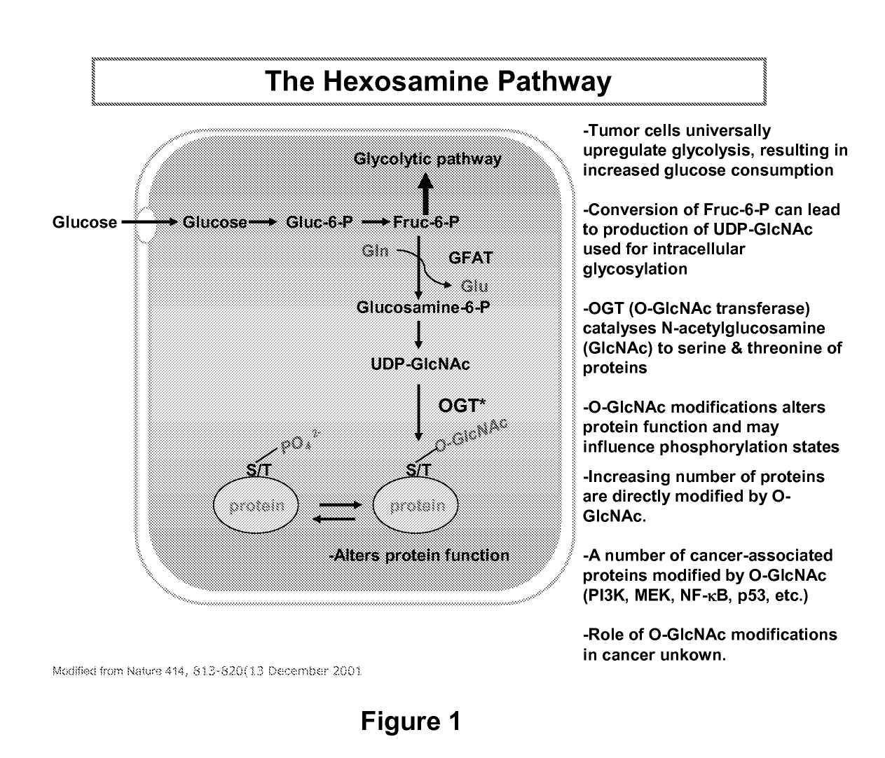

FIG. 1 shows a schematic of tumor cells which upregulate glycolysis, resulting in increased glucose consumption. Conversion of fructose-6-phosphate can lead to production of UDP-GlcNAc used for intracellular glycosylation. O-GlcNAc transferase (OGT) catalyses N-acetylglucosamine (GlcNAc) to serine and threonine of proteins. O-GlcNAc modifications alters protein function and may influence phosphorylation states. Increasing number of proteins are directly modified by O-GlcNAc.

A number of cancer-associated proteins are modified by O-GlcNAc, for example, PI3K, MEK, NF-κB, and p53. The role of O-GlcNAc modifications in cancer has not been previously determined. The present study shows that O-GlcNAc modifications occur in cancer cells, for example, epithelial-type cancers. Inhibition of O-GlcNAc modification is a potential mechanism to target tumor cells and for therapeutic treatment of cancer in a mammalian subjection.

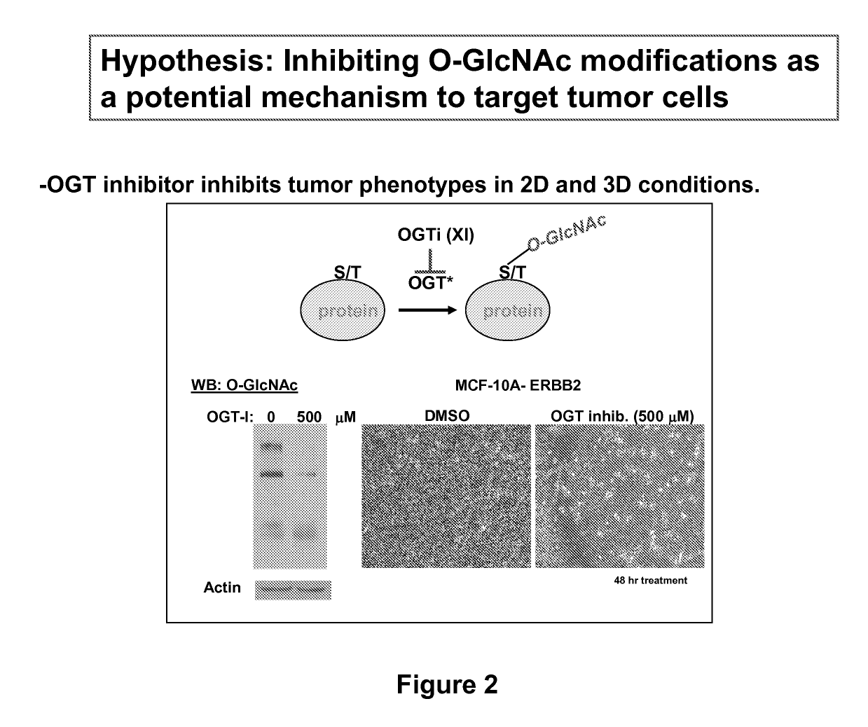

FIG. 2 outlines the rationale that inhibiting O-GlcNAc modifications as a potential mechanism to target tumor cells.

FIG. 3 shows that breast cancer cells upregulate hexosamine signaling pathway. Breast cancer cells contain elevated substrate UDP-GlcNAc . HPLC analysis of UDP-GlcNAc. Analyses were carried out on an AKTA purified HPLC. Separation of nucleotides and nucleotide sugars was performed on an Alltima C18 column. Peak identification and peak areas integration were performed using a mixture of standards injected before each analysis. Integrated peak areas were quantified for determination of nucleotide levels/mg of protein.

FIG. 4 shows that breast cancer cells upregulate hexosamine signaling pathway. Breast cancer cells contain higher O-GlcNAc modified proteins. Normal mammary epithelial cells (MCF-10A) cells and tumorigenic breast cancer cells were lysed after 48 hours of culturing and subjected to Western blot analysis to determine levels of proteins modified by O-GlcNAc (antibody specifically recognized proteins modified by O-GlcNAc).

FIG. 5 shows breast cancer cells upregulate hexosamine signaling pathway. Breast cancer cells overexpress the O-GlcNAc transferase OGT. Normal mammary epithelial cells (MCF-10A) cells and tumorigenic breast cancer cells were lysed after 48 hours of culturing and subjected to Western blot analysis with indicated antibodies.

FIG. 6 shows targeting OGT inhibits anchorage-independent growth of cancer cells. MCF-10A-NeuT cells were transfected with control or OGT siRNA. A. Cells were lysed and analysed by Western analysis with indicated antibodies. B. OGT knockdown cells show decreased O-GlcNAc modification. Control or OGT siRNA cells were treated overnight with indicated concentration of O-GlcNAcase inhibitor (9D) before being lysed and analyzed by Western analysis with indicated antibodies. C. OGT inhibition reduces colony formation in breast cancer cells. Control or OGT siRNA cells were placed in a soft agar assay for 14 days. Cell were stained with INT violet and colonies imaged in phase contrast or camera. D. OGT knockdown leads to three fold reduction in colony formation. Following the 14 days in soft agar, the number of colonies formed in each treatment was counted and averaged. The average of 4 trials are shown here.

FIG. 7 shows targeting OGT inhibits MCF-10-NeuT-mediated invasion and proliferation. MCF-10A-NeuT cells were transfected with control or OGT siRNA and then placed in 3D morphogenesis assay. At Day 6 cells were imaged in phase contrast and also fixed and analyzed using confocal microscopy with indicated antibodies. A. Ki67 is a marker for proliferating cells. Note OGT RNAi cells do not show invasive structures (protrusions) into matrigel. B. Acini were stained with adhesion receptors integrin α5 and α6.

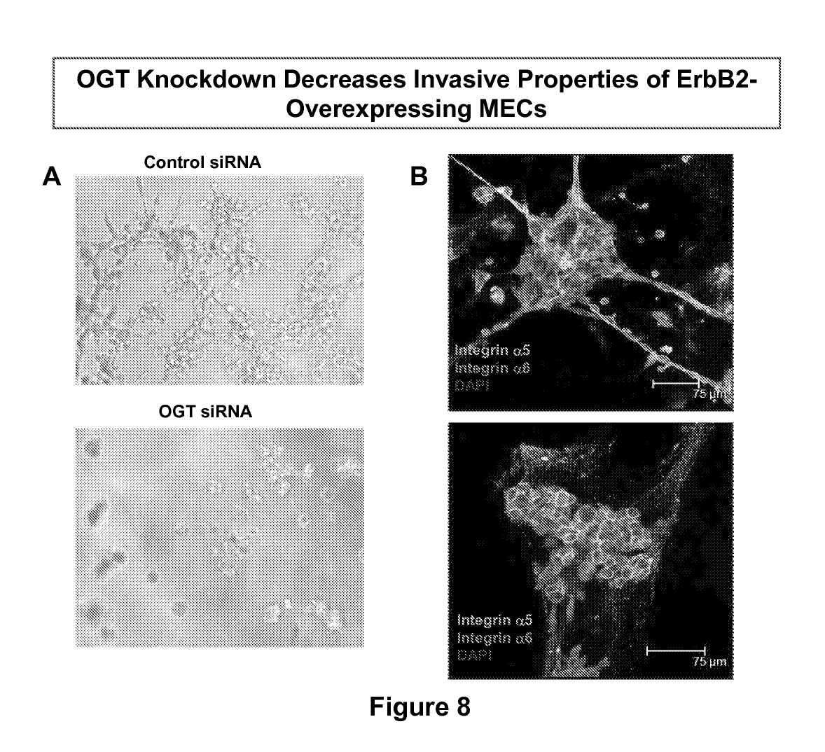

FIG. 8 shows targeting OGT inhibits MCF-10-NeuT-mediated invasion and proliferation. MCF-10A-NeuT cells were transfected with control or OGT siRNA and then placed in 3D morphogenesis assay. At Day 6 cells were imaged in phase contrast and also fixed and analyzed using confocal microscopy with indicated antibodies. A. Ki67 is a marker for proliferating cells. Note OGT RNAi cells do not show invasive structures (protrusions) into matrigel. B. Acini were stained with adhesion receptors integrin α5 and α6.

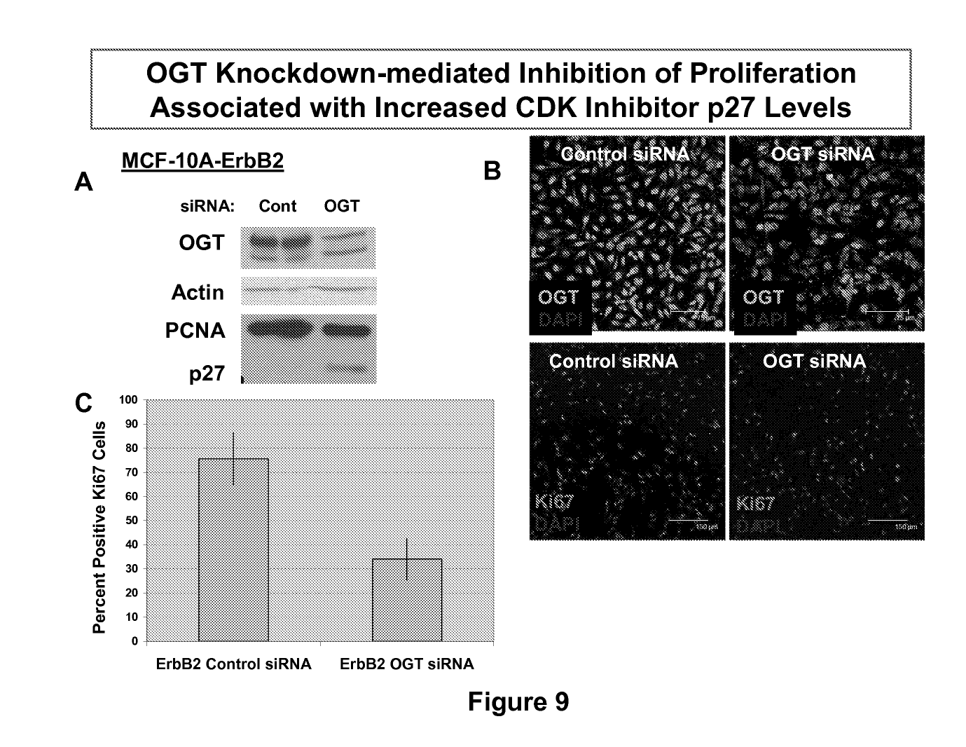

FIG. 9 shows OGT knockdown-mediated inhibition of proliferation is associated with induction of CDK inhibitor p27.MCF-10A-NeuT cells were transfected with control or OGT siRNA. A. Cells were lysed and subjected to Western blot analysis with indicated antibodies. B. At 48 hours, cells were replated on coverslips and fixed 24 hours and stained to analyze with confocal microscopy with indicated antibodies. C. Proliferating (Ki67 positive) cells were counted. In each trail 400 cells of each treatment were counted and the number of Ki67 positive cells recorded. The average of 4 experiments is shown here.

FIG. 10 shows OGT inhibitor (ST060266) blocks growth and invasion on MCF-10A-NeuT cells. A. MCF-10A-NeuT cells were treated with vehicle control (DMSO) or 500 μM OGT inhibitor (ST060266, TimTec LLC) for 48 hours. Cells were lysed and proteins analyzed by Western blot to determine total O-GlcNAc levels. B. MCF-10A-NeuT cells were placed in 3D basement membrane culture and treated with OGT inhibitor or vehicle control. At day 8, phase images of the acini were taken and cells were fixed and stained for confocal microscopy with indicated antibodies.

FIG. 11 shows OGT inhibitor (ST060266) blocks growth and invasion on MCF-10A-NeuT cells. MCF-10A-NeuT cells were placed in transwell invasion slides in the presence of DMSO or 500 μM OGT inhibitor (ST060266, TimTec LLC) for 24 hrs. Cells invading matrigel and membrane were stained with DAPI and counted.

FIG. 12 shows targeting O-GlcNAc modifications has no effect on growth or differentiation of normal mammary epithelial cells. A. MCF-10A cells were transfected with control or OGT siRNA. Control and OGT siRNA transfected cells were treated with DMSO or O-G1cNAcase inhibitor 9D (100 μM) and proteins modified by O-GlcNAc were analyzed by Western blotting. B. Control and OGT siRNA transfected cells were placed in 3D morphogenesis assay and images were taken at indicated day.

FIG. 13: shows that targeting O-GlcNAc modifications has no effect on growth or differentiation of normal mammary epithelial cells. MCF-10A cells were treated with DMSO or 500 μM OGT inhibitor (ST060266, TimTec LLC) and placed in 3D morphogenesis assay and imaged with confocal microscopy with indicated antibodies.

The present results demonstrate that cancer cells upregulate hexosamine signaling pathway including elevating UDP-GlcNAc levels, increased O-GlcNAc modifications and OGT levels. Decreasing O-GlcNAc modifications by targeting OGT, with RNAi or a specific inhibitor, inhibits tumor phenotypes including colony formation in soft agar, cell growth, and cell invasion. Inhibiting OGT in tumor cells decreases proliferation markers PCNA and Ki-67 and increases CDK inhibitor p27. Targeting OGT has minimal effect of growth and differentiation of normal mammary epithelial cells.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

What is claimed:

1. A method for treating neoplastic disease in a mammalian subject comprising
administering to the subject an inhibitor of O-GlcNAc transferase in an amount effective to reduce or eliminate the neoplastic disease in the mammalian subject.

2. The method of claim 1 wherein the neoplastic disease is an epithelial-derived cancer.

3. The method of claim 2 wherein the neoplastic disease is breast cancer, ovarian cancer, gastric cancer, lung cancer, liver cancer, prostate cancer, skin cancer or salivary cancer.

4. The method of claim 1 wherein the inhibitor is a small chemical molecule, an antibody, an antisense nucleic acid, short hairpin RNA, or short interfering RNA.

5. A method for diagnosing a risk factor for a neoplastic disease in a mammalian subject comprising,
contacting cells or tissue from the subject with an assay system for detecting a cellular component upregulated in a hexosamine signaling pathway in the cell or tissue, and
comparing the upregulation of the hexosamine signaling pathway in the cell or tissue to normal function of the hexosamine signaling pathway in the cell or tissue.

6. The method of claim 5 wherein the upregulated hexosamine signaling pathway is measured as elevated UDP-GlcNAc levels in the cell or tissue, increased O-GlcNAc modification of proteins, or increased activity of O-GlcNAc transferase enzyme, or elevated levels of O-GlcNAc transferase gene expression or O-GlcNAc transferase protein in the cell or tissue.

7. The method of claim 5 wherein the assay system comprises an antibody or a nucleic acid.

8. The method of claim 5 wherein the neoplastic disease is an epithelial-derived cancer.

9. The method of claim 8 wherein the neoplastic disease is breast cancer, ovarian cancer, gastric cancer, lung cancer, liver cancer, prostate cancer, skin cancer or salivary cancer.

10. A method for identifying a test compound which inhibits a hexosamine signaling pathway in a cell or tissue comprising:
contacting the test compound with a cell-based assay system comprising a cell expressing O-GlcNAc transferase, and capable of catalyzing N-acetylglucosamine attachment to a serine or threonine residue of a protein, and
detecting an effect of the test compound on inhibition of O-GlcNAc transferase activity or O-GlcNAc transferase gene expression, effectiveness of the test compound in the assay being indicative of inhibition of the hexosamine signaling pathway activity in the cell or tissue.

11. The method of claim 10 wherein the cell is a neoplastic cell.

12. The method of claim 11 wherein the cell is a mammary epithelial cell expressing ErbB2, BT20 tumor cell, MDA-MB-231 tumor cell, MDA-MB-453 tumor cell, MDA-MB-468 tumor cell or SKBR3 tumor cell.

13. The method of claim 11 wherein inhibition of hexosamine signaling is measured by inhibition of a tumor cell phenotype.

14. The method of claim 13 wherein the tumor cell phenotype is cell growth, colony formation in soft agar, or cell invasion.

15. The method of claim 14 wherein the test compound inhibits cell growth, colony formation, or cell invasion growth of the neoplastic cell, and the test compound does not inhibit cell growth or differentiation of a non-neoplastic cell.

16. The method of claim 15 wherein the neoplastic cell is an epithelial tumor cell and the non-neoplastic cell is an epithelial cell.

17. The method of claim 14 wherein the test compound effect on the neoplastic cell decreases cell proliferation markers PCNA or Ki-67, or increases CDK inhibitor p27, or a combination thereof

18. The method of claim 10 wherein the test compound is a small chemical molecule, an antisense nucleic acid, short hairpin RNA, or short interfering RNA.
